(19) United States
(12) Patent Application Publication
(10) Pub. No.: US 2008/0153853 A1
(43) Pub. Date: Jun. 26, 2008

(54) USE OF MTKI 1 FOR TREATING OR PREVENTING BRAIN CANCER

(76) Inventors: Eddy Jean Edgard FREYNE, Rumst (BE); Michel Marie Francois Janicot, Mol (BE); Martin John Page, Schilde (BE); Timothy Pietro Suren Perera, Geel (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/875,350

(22) Filed: Oct. 19, 2007

Related U.S. Application Data
(60) Provisional application No. 60/863,162, filed on Oct. 27, 2006.
(60) Provisional application No. 60/976,168, filed on Sep. 28, 2007.

(51) Int. Cl. A61K 31/519; A61P 35/00

(52) U.S. Cl. 514/257

(57) ABSTRACT

The present invention is concerned with the finding that some the macrocyclic quinazoline derivative 4,6-ethanediylidenepyrimido[4,5-b][6,1,12]benzoxadiazacyclo-pentadecine, 17-bromo-8,9,10,11,12,13,14,19-octahydro-20-methoxy-13-methyl-, described as compound 22 in PCT publication W02004/105765, is useful in the manufacture of a medicament for the treatment or prevention of a primary brain cancer or brain metastasis. It accordingly provides methods for treating, preventing, delaying or mitigating brain cancer, or for preventing or slowing proliferation of cells of brain origin.

### CROSS-REFERENCE TO RELATED APPLICATION

This application claims priority to U.S. Provisional Application for Patent No. 60/863,162, filed Oct. 27, 2006, and U.S. Provisional Application for Patent No. 60/976,168 filed Sep. 28, 2007, the entire disclosures of which are hereby incorporated in their entirely.

### FIELD OF THE INVENTION

The present invention is concerned with the finding that the macrocyclic quinazoline derivative 4,6-ethanediylidenepyrimido[4,5-b][6,1,12]benzoxadiazacyclo-pentadecine, 17-bromo-8,9,10,11,12,13,14,19-octahydro-20-methoxy-13-methyl, described as compound 22 in PCT publication WO2004/105765, is useful in the manufacture of a medicament for the treatment of a primary brain cancer or treatment or prevention of brain metastasis. It accordingly provides methods for treating, preventing, delaying or mitigating brain metastasis and treating, delaying or mitigating primary brain cancer.

### BACKGROUND OF THE INVENTION

“Brain cancer” means (1) any abnormally increased proliferation of any type of neuronal cell, hereinafter also referred to as primary brain cancer, or (2) any other cancer that has metastasized into the central nervous system (CNS), hereinafter also referred to as brain metastases.

Most neuronal cells—that is cells, that comprise or are found in the CNS, including, for example, neurons, microglia, and astrocytes—are “terminally differentiated”, meaning that they no longer possess the ability to complete the cell cycle. (Kornblith et al., (1986), Cancer: Principles and Practice of Oncology, 2^{nd} Ed., DeVita, V., Hellman, S., Rosenberg, S, eds., J. B. Lippincott Company, Philadelphia, Chapter 41: Neoplasms of the Central Nervous System). Even if neuronal cells would enter the cell cycle, they are usually unable to complete the process as they would undergo apoptosis (cell death) (Multani, A. S., et al., Neoplasia 2(4), 339-45 (2000)). Only in those cases where neuronal cells lose the protective ability to undergo apoptosis, primary brain cancers may occur. Examples of primary brain cancers include, but are not limited to, neuroma, astrocytoma, neuroblastoma, glioma, meningioma, oligodendroglioma, medulloblastoma, spinal cord tumor and schwannoma.

Gliomas comprise about 60% of all primary CNS tumors and usually occur in the cerebral hemisphere of the brain, but may be found in other areas such as the optic nerve, brain stem or cerebellum. Gliomas are classified into groups according to the type of glial cell from which they originate (Kornblith et al., (1986), Cancer: Principles and Practice of Oncology, 2^{nd} Ed., DeVita, V., Hellman, S., Rosenberg, S, eds., J. B. Lippincott Company, Philadelphia, Chapter 41: Neoplasms of the Central Nervous System). The most common types of glioma are astrocytomas. These tumors develop from star-shaped glial cells called astrocytes. Astrocytomas are assigned to grades according to their malignancy. Low-grade astrocytomas, also known as grade I and II astrocytomas, are the least malignant, grow relatively slow and can often be completely removed using surgery. Mid-grade astrocytomas, also known as grade III astrocytomas, grow more rapidly and are more malignant. Grade III astrocytomas are treated with surgery followed by radiation and some chemotherapy. High-grade astrocytomas, also known as grade IV astrocytomas, grow rapidly, invade nearby tissue, and are very malignant. Grade IV astrocytomas are usually treated with surgery followed by a combination of radiation therapy and chemotherapy. Glioblastoma multiforme are grade IV astrocytomas, which are among the most malignant and deadly primary brain tumors (Id). While the same surgical techniques and principles have applied to treating glioblastoma multiforme and less malignant brain tumors, total removal of a glioblastoma multiforme tumor has been more difficult to achieve.

The difference in malignancy is also reflected in the prognosis for a patient having a primary brain tumor. While a person treated for a grade I astrocytoma can commonly survive 10 years or more without recurrence, the mean length of survival for a patient with a grade IV astrocytoma tumor is 15 weeks after surgical treatment. Because of the high malignant-growth potential of grade IV astrocytoma tumors, only 5% of patients have survived for 1 year following surgical treatment alone, with a near 0% survival rate after 2 years. Radiation treatment in combination with surgical treatment increases the survival rate to about 10% after 2 years of treatment; however, virtually no patients survive longer than 5 years (Id).

While a treatment regimen of surgery, radiation therapy and chemotherapy offers the opportunity for a modestly increased lifespan for patients with a grade IV astrocytoma brain tumor, the risks associated with each method of treatment are many. The benefits of treatment are minimal, and treatment can significantly decrease the quality of the patient's brief remaining lifespan. Accordingly, there remains a clear need in the art for primary brain cancer prevention and treatment methods that overcome the disadvantages of the above-mentioned traditional approaches, in particular as CNS tumors represent the most common group of solid tumors in young patients. Brain tumors are the second-leading cause of cancer-related deaths in children, accounting for approximately 25% of all such deaths, and with the current therapies a majority of these children die within the first year of diagnosis.

Compared to primary brain tumors the incidence of brain metastases is much higher. Approximately 100,000 patients have symptomatic intracranial metastases in the USA annually and according to autopsy studies, a quarter of cancer patients have tumor metastases (Newton, H. B., et al., J. Neurooncol. 61, 35-44 (2003)). The brain metastasis results from a primary tumor elsewhere in the body, include but are not limited to, for example, lung cancer (both small cell and non-small cell), breast cancer, colorectal cancer, prostate cancer, melanoma and pancreatic cancer. In particular in patients with metastatic breast cancer, the incidence of brain metastases is diagnosed at a rate of 10 to 20% (Tyson, R. M. et al., Therapy 3(1), 97-112 (2006)). Breast cancer is the second leading cause of cancer-related deaths in women and almost all deaths from breast cancer are due to metastatic disease with brain metastasis found in 30% of patients at autopsy.

The standard mode of treatment comprise surgical resection, chemotherapy and radiation treatment in particular whole-brain radiotherapy (WBRT) and stereotactic radiosurgery (SRS) or a combination thereof. Surgery for brain metastasis can improve survival, especially in patients with single lesions. However, surgery may not be possible in the face of multiple lesions, surgically inaccessible lesions or patients with an inability to tolerate surgery. In breast cancer, 50% of the patients with brain cancer metastasis have multiple metastases, making them less suitable as surgical candidates. WBRT may improve median survival over no treatment, and as an adjuvant to surgery it reduces the recurrence rate and chances of dying a neurological death, but it does not change survival or level of function. SRS provides a method of treating brain metastasis that may be surgically unresectable, either by location or patient condition. SRS provides an improvement in quality of life but does not provide survival benefit, except in patients with a single metastasis. The use of chemotherapy in the treatment of brain metastasis is hampered by the inability of large molecular-weight compounds, thus restricting most chemotherapy agents, to cross the blood-brain barrier. This is reflected in the fact that chemosensitive tumors, such as most metastatic breast carcinomas, often show complete systemic responses to chemotherapy concomitant with tumor progression in the brain. However, this initial reluctance to the use of chemotherapy in the treatment of brain metastasis did change over the recent years based on findings in animal models and human brain tumor autopsies, that metastatic lesions result in an impaired blood-brain barrier and by virtue of this, chemotherapy drugs can invariably enter into the tumor. Radiotherapy in conjuction with chemotherapy has evolved into the first-line approach in the treatment of brain metastases.

However, there are serious limitations and dangers associated with all of the current methods of treatment. Radiotherapy, which often attempts to deliver highly destructive doses of ionizing radiation through the normal tissues of the body in an attempt to preferentially kill highly specific and often imperfectly defined areas of cancerous tissue, can have serious and significant side effects due to the destruction of normal nervous system or other tissues of the body, leading amongst others, to memory loss and personality alterations (infra). Chemotherapy, which attempts to preferentially kill cancerous cells instead of normal cells through the diverse administration of chemical agents or drugs to the tissues of the body, is limited in efficacy by the chemical agents currently available and can lead to toxic and unintended side effects on normal tissue. Surgery, which attempts to mechanically destroy or intervene in the progression of cancer, can also lead to serious side effects or consequences as a result of mechanical trauma or destruction of normal tissue. Some of the problems associated with the above approaches are (i) adverse side effects including alterations of intelligence, learning ability, memory, motor function, consciousness, and emotion; (ii) re-emergence of the tumor within three to five years of treatment due to development of resistance to these therapies; (iii) death due to the ineffectiveness of such treatment.

From the foregoing, it will be apparent that there exists an urgently compelling, yet unsatisfied need to develop chemotherapeutic agents that can cross the blood-brain barrier in an amount effective to reduce growth and/or neoplastic spread of the cancer in the central nervous system.

### SUMMARY OF THE INVENTION

The invention is directed in part to methods of treating or preventing brain cancer, and/or the treatment or prevention of brain metastasis utilizing certain compounds described in WO 2004/105765, the disclosure of which is hereby incorporated by reference in its entirety.

In one embodiment, the present invention provides the use of the macrocyclic quinazoline derivative 4,6-ethanediylidenepyrimido[4,5-b][6,1,12]benzoxadiazacyclo-pentadecine, 17-bromo-8,9,10,11,12,13,14,19-octahydro-20-methoxy-13-methyl, described as compound 22 in PCT publication WO2004/105765, in the manufacture of a medicament for the treatment or prevention of a primary brain cancer or brain metastasis.

In related embodiment, the invention provides a method of inhibiting metastatic spread of a cancer to the central nervous system, in a mammalian subject comprising administering to a mammalian subject suspected of having metastatic cancer a compound of the invention, in an amount effective to inhibit metastatic spread of the cancer to the central nervous system; and a method for treating brain cancer comprising administering to a mammalian subject diagnosed with a cancer a composition comprising a compound of the invention, in an amount effect to reduce growth or neoplastic spread of the brain cancer/metastasis. It will be appreciated that any reduction in the rate of cancer growth or spread (which can prolong life and quality of life) is indicative of successful treatment. In preferred embodiments, cancer growth is halted completely. In still more preferred embodiments, cancers shrink or are eradicated entirely. Preferred subjects for treatment are human subjects, but other animals, especially murine, rat, canine, bovine, porcine, primate, and other model systems for cancer treatment, are contemplated.

In one variation of the foregoing methods of treatment, the compounds are administered along with a second cancer therapeutic agent. The second agent can be any chemotherapeutic agent, radioactive agent, irradiation, nucleic acid encoding a cancer therapeutic agent, antibody, protein, and/or other anti-lymphangiogenic agent or an anti-angiogenic agent. The second agent may be administered before, after, or concurrently with the compounds of the invention.

In one variation, the subject to be treated has been diagnosed with an operable tumor, and the administering step is performed before, during, or after the tumor is resected from the subject. Compound treatment in conjunction with tumor resection is intended to reduce or eliminate regrowth of tumors from cancer cells that fail to be resected.

Stated more generically, the invention provides a method of treating a brain cancer, and the treatment or prevention of brain metastasis comprising the step of administering to a mammal (including, but not limited to humans, rats, canines, bovines, porcines, and primates) in need thereof a compound of the invention.

### BRIEF DESCRIPTION OF THE DRAWINGS

FIG. 1: Effect of 4,6-ethanediylidenepyrimido[4,5-b][6,1,12]benzoxadiazacyclopentadecine, 17-bromo-8,9,10,11,12,13,14,19-octahydro-20-methoxy-13-methyl (Compound 1) on s.c A431 tumor growth. Gray bar indicates the treatment period with Compound 1,i.e. p.o., QD×14. Black arrows indicate treatment with the reference compound BCNU (Carmustin), i.e. i.v., Q14D×2. Grey arrow—Day 18. On day 18 the median subcutaneous (s.c.) tumor volumes were statistically analysed.

FIG. 2: Statistical analysis of the median subcutaneous (s.c.) tumor volumes of the A431 cells. Gray bar indicates the treatment period with Compound 1, i.e. p.o., QD×14.

FIG. 3: Kapalan Myer survival curves showing the survival of rats bearing intracranial and subcutaneous tumors. On day 91 some surviving animals start to be sacrificed due to extensive s.c tumor burden.

### DETAILED DESCRIPTION OF THE INVENTION

WO-2004/105765 describes the preparation, formulation and pharmaceutical properties of macrocyclic quinazoline derivatives of formula (I) as multi targeted kinase inhibitors (MTKIs).

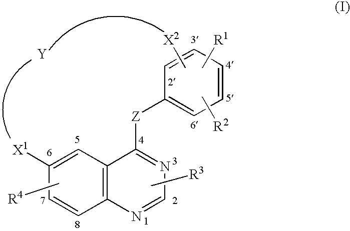

It has now been found that one compound in the aforementioned class, i.e 4,6-ethanediylidenepyrimido[4,5-b][6,1,12]benzoxadiazacyclo-pentadecine, 17-bromo-8,9,10,11,12,13,14,19-octahydro-20-methoxy-13-methyl, described as compound 22 in the aforementioned PCT publication, herein also referred to as MTKI 1 and/or Compound 1, has clinical activity in brain cancer models and accordingly provide the use of this compound for the preparation of a pharmaceutical composition for treating brain cancer, including primary brain cancers and brain metastases as defined hereinbefore.

Accordingly, in one aspect the present invention provides the use of 4,6-ethanediylidenepyrimido[4,5-b][6,1,12]benzoxadiazacyclo-pentadecine, 17-bromo-8,9,10,11,12,13,14,19-octahydro-20-methoxy-13-methyl or a pharmaceutically acceptable acid or base addition salt thereof, in the manufacture of a medicament for the treatment or prevention of brain cancer.

A further aspect of the present invention is directed to a method for the treatment or prevention of brain cancer in a mammalian subject, comprising administering a therapeutically effective amount of 4,6-ethanediylidenepyrimido[4,5-b][6,1,12]benzoxadiazacyclo-pentadecine, 17-bromo-8,9,10,11,12,13,14,19-octahydro-20-methoxy-13-methyl or a pharmaceutically acceptable acid or base addition salt thereof, to a mammalian subject in need of such treatment.

The pharmaceutically acceptable acid or base addition salts as mentioned hereinabove are meant to comprise the therapeutically active non-toxic acid and non-toxic base addition salt forms which MTKI 1 is able to form. The basic properties can be converted in their pharmaceutically acceptable acid addition salts by treating said base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The acidic properties may be converted in their pharmaceutically acceptable base addition salts by treating said acid form with a suitable organic or inorganic base. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which MTKI 1 is able to form. Examples of such forms are e.g. hydrates, alcoholates and the like.

In particular, the present invention is concerned with a use of the dihydrobromide salt of 4,6-ethanediylidenepyrimido[4,5-b][6,1,12]benzoxadiazacyclo-pentadecine, 17-bromo-8,9,10,11,12,13,14,19-octahydro-20-methoxy-13-methyl, i.e., 17-bromo-8,9,10,11,12,13,14,19-octahydro-20-methoxy-13-methyl-4,6-ethanediylidenepyrimido [4,5-b][6,1,12]benzoxadiazacyclopentadecine dihydrobromide, in any of the aforementioned uses for MTKI 1.

In a further embodiment, the present invention provides the use of the aforementioned MTKI 1 for the preparation of a pharmaceutical composition for the treatment and/or prevention of brain cancers.

The present invention also concerns a method of treating and/or preventing brain cancer in a mammal, comprising the step of administering a therapeutically effective amount of the aforementioned MTKI 1 to said mammal.

In a further embodiment, the present invention provides the use of the aforementioned MTKI 1 for the preparation of a pharmaceutical composition for the treatment and/or prevention of brain metastasis.

The present invention also concerns a method of treating and/or preventing brain metastasis in a mammal, comprising the step of administering a therapeutically effective amount of the aforementioned MTKI 1 to said mammal.

Accordingly, in a further aspect, the most preferred compounds for use in accordance with the present invention are those selected from the group consisting of compounds having the following structure:

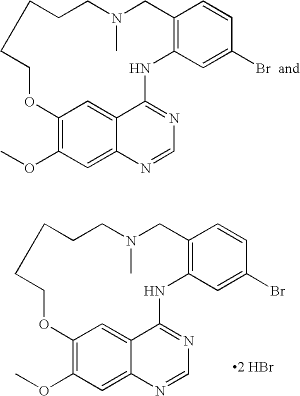

The compounds according to the invention can be prepared and formulated into pharmaceutical compositions by methods known in the art and in particular according to the methods described in the published patent specification WO-2004/105765 mentioned herein and incorporated by reference.

A suitable preparation of the preferred compound used in this invention, taken from WO-2004/105765, follows:

### EXAMPLE 1

### a) Preparation of 1-pentanol, 5-[[(4-bromo-2-nitrophenyl)methyl]amino]-(Intermediate 1)

A solution of 4-bromo-2-nitro-benzaldehyde,(0.013 mol), 5-amino-1-pentanol (0.013 mol) and titanium, tetrakis (2-propanolato) (0.014 mol) in EtOH (15 ml) was stirred at RT for 1 hour, then the reaction mixture was heated to 50° C. and stirred for 30 min. The mixture was cooled to RT and NaBH₄ (0.013 mol) was added portionwise. The reaction mixture was stirred overnight and then poured out into ice water (50 ml). The resulting mixture was stirred for 20 min., the formed precipitate was filtered off (giving Filtrate (I)), washed with H₂O and stirred in DCM (to dissolve the product and to remove it from the Ti-salt). The mixture was filtered and then the filtrate was dried (MgSO₄) and filtered, finally the solvent was evaporated. Filtrate (I) was evaporated until EtOH was removed and the aqueous concentrate was extracted 2 times with DCM. The organic layer was separated, dried (MgSO₄), filtered off and the solvent was evaporated, yielding 3.8 g (93%) of intermediate 1.

### EXAMPLE 2

### a) Preparation of 1-pentanol, 5-[[(4-bromo-2-nitrophenyl)methyl]methylamino]-(Intermediate 2)

A solution of intermediate 50 (0.0047 mol), formaldehyde (0.025 mol) and titanium, tetrakis (2-propanolato) (0.0051 mol) in EtOH (150 ml) was heated to 50° C. and stirred for 1 hour, then NaBH₄ (0.026 mol) was added portionwise at RT. The reaction mixture was stirred overnight and then quenched with water (100 ml). The resulting mixture was stirred for 1 hour; the formed precipitate was filtered off and washed. The organic filtrate was concentrated, then the aqueous concentrate was extracted with DCM and dried. The solvent was evaporated and the residue was filtered over silica gel (eluent: DCM/CH₃OH from 98/2 to 95/5). The product fractions were collected and the solvent was evaporated, yielding 0.5 g of intermediate 2.

### b) Preparation of 1-pentanol, 5-[[(4-bromo-2-nitrophenyl)methyl]methylamino]-, acetate (ester) (Intermediate 3)

A solution of intermediate 2 (0.0015 mol) and pyridine (0.015 mol) in acetic anhydride (8 ml) was stirred overnight at RT, then the solvent was evaporated and co-evaporated with toluene, yielding intermediate 3.

### c) Preparation of 1-pentanol, 5-[[(2-amino-4-bromophenyl)methyl]methylamino]-, acetate (ester) (Intermediate 4)

A mixture of intermediate 3 (0.0015 mol) in THF (50 ml) was hydrogenated with Pt/C 5% (0.5 g) as a catalyst in the presence of thiophene solution (0.5 ml) [H179-034]. After uptake of H₂ (3 equiv.), the catalyst was filtered off and the filtrate was evaporated, yielding 0.5 g of intermediate 4.

### d) Preparation of 6-quinazolinol, 4-[[2-[[[5-(acetyloxy)pentyl]methylamino]methyl]-5-bromophenyl]amino]-7-methoxy-, acetate (ester) (Intermediate 5)

A mixture of intermediate 4 (0.0015 mol) and 4-chloro-7-methoxy-6-quinazolinol acetate (ester) (0.0015 mol) in 2-propanol (30 ml) was heated to 80° C. and the reaction mixture was stirred for 1 day. The solvent was evaporated under reduced pressure and the residue was used as such in the next reaction step, yielding 0.83 g of intermediate 5.

### e) Preparation of 6-quinazolinol, 4-[[5-bromo-2-[[(5-hydroxypentyl)methylamino]methyl]phenyl]amino]-7-methoxy-(Intermediate 6)

A solution of intermediate 5 (0.0015 mol) in methanol (25 ml) was stirred at RT and a solution of K₂CO₃ (0.003 mol) in H₂O (2.5 ml) was added, then the reaction mixture was heated to 60° C. and stirred for 18 hours. The solvent was evaporated and H₂O (20 ml) was added, then the mixture was neutralized with acetic acid and the formed precipitate was filtered off. The filtrate was concentrated under reduced pressure and the concentrate was extracted with DCM, filtered, then dried (MgSO₄) and the mixture was concentrated under reduced pressure, yielding 0.5 g (70%) of intermediate 6.

### EXAMPLE 3

### a)Preparation of 4,6-ethanediylidenepyrimido[4,5-b][6,1,12]benzoxadiazacyclo-pentadecine, 17-bromo-8,9,10,11,12,13,14,19-octahydro-20-methoxy-13-methyl-(compound MTKI1)

A solution of intermediate 6 (0.0011 mol) in THF (50 ml) was stirred at RT and tributylphosphine (0.0016 mol) was added, then 1,1′-(azodicarbonyl)bis-piperidine (0.0016 mol) was added and the reaction mixture was stirred for 2 hours. The solvent was evaporated until ⅓ of the initial volume. The resulting precipitate was filtered off and washed. The filtrate was evaporated and the residue was purified by RP high-performance liquid chromatography. The product fractions were collected and the organic solvent was evaporated. The aqueous concentrate was extracted 2 times with DCM and the organic layer was dried (MgSO₄), then filtered off. The solvent was evaporated and the residue was dried (vac.) at 50° C., yielding 0.004 g (0.8%) of compound MTKI1.

To prepare the aforementioned pharmaceutical compositions, a therapeutically effective amount of the particular compound, optionally in addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous, or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions (including nanosuspensions), syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable solutions containing compounds of formula (I) may be formulated in an oil for prolonged action. Appropriate oils for this purpose are, for example, peanut oil, sesame oil, cottonseed oil, corn oil, soy bean oil, synthetic glycerol esters of long chain fatty acids and mixtures of these and other oils. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment. As appropriate compositions for topical application there may be cited all compositions usually employed for topically administering drugs e.g. creams, gels, dressings, shampoos, tinctures, pastes, ointments, salves, powders and the like. Application of said compositions may be by aerosol, e.g. with a propellent such as nitrogen, carbon dioxide, a freon, or without a propellent such as a pump spray, drops, lotions, or a semisolid such as a thickened composition which can be applied by a swab. In particular, semisolid compositions such as salves, creams, gels, ointments and the like will conveniently be used.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Preferably, a therapeutically effective amount of the pharmaceutical composition comprising a compound according to the invention, is administered orally or parenterally. Said therapeutically effective amount is the amount that effectively prevents metastasis and/or growth or reduces the size of a variety of neoplastic disorders or cell proliferative disorders (supra) in patients. On the basis of the current data, it appears that a pharmaceutical composition comprising a compound of the present invention, and in particular 4,6-ethanediylidenepyrimido[4,5-b][6,1,12]benzoxadiazacyclo-pentadecine, 17-bromo-8,9,10,11,12,13,14,19-octahydro-20-methoxy-13-methyl (MTKI 1) as the active ingredient can be administered orally in an amount of from 10 mg to several (1 to 5) grams daily, either as a single dose or subdivided into more than one dose, including, e.g. two, three or even four times daily. A preferred amount ranges from 500 to 4,000 mg daily. A particularly, preferred dosage for such a compound is in the range of 750 mg to 3,000 mg daily. It will be appreciated that the amount of a compound according to the present invention, also referred to here as the active ingredient, which is required to achieve a therapeutic effect will, of course, vary with, the route of administration, the age and condition of the recipient, and the particular disorder or disease being treated. The optimum dosage amounts and regimen can be readily determined by those skilled in the art using conventional methods and in view of the information set out herein. This treatment can be given either continuously or intermittently, including, e.g. but not limited to, cycles of 3-4 weeks with treatment given for 1-21 days per cycle or other schedules shown to be efficacious and safe.

One illustrative formulation is as follows:

### EXAMPLE 4

### Formulation

The product MTKI1 can be prepared as a 10-mg/mL oral solution, pH 2. It contains an excipient, Captisol® (chemical name: sulfobutyl ether-β-cyclodextrin, SBE-β-CD), citric acid, Tween® 20, HCl, and NaOH in purified water. The formulation can be stored refrigerated (2-8° C.; 36-46° F.) and allowed to warm to room temperature for maximally 1 hour prior to dose preparation.

The product MTKI1 can also be prepared as 50-mg, 100-mg and 300-mg oral immediate release capsules, containing the active chemical entity MTKI1, lactose monohydrate (200 mesh), sodium lauryl sulphate and magnesium stearate in hard gelatin capsules, sizes 3, 4 and 00, respectively. The capsules may also contain any or all of the following ingredients: gelatin, red iron oxide and titanium oxide.

The above MTKI of the present invention may be used in combination with one or more other cancer treatments. Such combinations could encompass any established antitumor therapy, such as, but not limited to, chemotherapies, irradiation, and target based therapies such as antibodies and small molecules (including, but not limited to Temozolomide or BCNU). These therapies may be combined in systemic therapy, or local instillation/administration (e.g. intrathecally), depending on optimum efficacy/safety requirements.

The MTKI 1 (e.g., Compound 1) and the further anti-cancer agent may be administered simultaneously (e.g. in separate or unitary compositions) or sequentially in either order. In the latter case, the two compounds will be administered within a period and in an amount and manner that is sufficient to ensure that an advantageous additive or synergistic effect is achieved. It will be appreciated that the preferred method and order of administration and the respective dosage amounts and regimens for each component of the combination will depend on the particular MTKI and further anti-cancer agents being administered, their route of administration, the particular tumor being treated and the particular host being treated. The optimum method and order of administration and the dosage amounts and regimen can be readily determined by those skilled in the art using conventional methods and in view of the information set out herein.

### EXPERIMENTAL DATA

The unique physico-chemical properties of MTKI 1 has resulted in an extremely favourable tissue distribution profile including the ability to cross the intact blood brain barrier whilst still retaining good cellular activity and oral bioavailability. Here, we demonstrate that this preferential tissue distribution to the brain results in significant anti tumoral activity using experimental models of brain metastases.

A431 (ATCC, Rockville, Md. USA) vulval carcinoma cells were stereo-tactically injected into athymic nude rats and mice. Tumor growth delay was followed by using animal survival as readout in the rat study or using MRI imaging in the case of the mouse study.

Our data demonstrate that MTKI 1 potently delays tumor growth, leading to increased survival in experimental models of brain metastases.

### Methods

### Brain Models

### Stereotaxic Injection of Cells in the Brain of Rats

Forty (40) rats (Wistar, BDIX or Sprague Dawley) in part I and 40 nude rats were sterotaxically injected with cells at D0. For stereotaxic injection of tumor cells, rats were anesthetised by an intramuscular injection of a Ketamine (Ketamine500®, Ref 043KET204, Centravet, France) and Xylazine (Rompun®, Ref 002ROM001, Centravet, France) mixture (2/1, v/v, 70 and 15 mg/kg, respectively). 1×10⁵ A431 Cells were stereotaxically injected using 4 independant stereotaxic apparatus (Kopf Instrument, Germany and Stoelting Company, USA) in the right frontal lobe with 1×10⁵ tumor cells re-suspended in 5 μl of RPMI medium. Five μl of the cell suspension was injected according to SOP No TEC-083/001 at 0.5 μl/min. After cells injection, rats were observed during 1 hour.

### Subcutaneous Injection of Cells in Rats

The same day of the stereotaxic injection, rats were injected subcutaneously with A431 cells at D0. The tumor was obtained in rats by subcutaneous (SC) injection of 1×10⁷ A431 cells in RPMI medium (200 μl) in their right flank. For subcutaneous injection of tumor cells, rats were anesthetised by inhalation of Isoflurane Forene (Minerve, Bondoufle, France).

MTKI 1 (Compound 1 in the figures hereinafter) was dissolved in a solution of 20% Hydroxypropyl-β-cyclodextrine (20% β-HP-Cyclodextrin, pH 4.0) and given by oral gavage, daily for a period of fourteen days starting four days post inoculation of the cells, i.e. from day 4-17. Vehicle treated animals only received the β-HP-Cyclodextrin solution.

The reference compound BCNU (Carmustin) was dosed intravenously on day 4 and day 18.

### Results

### Brain Models

### Subcutaneous Injection of Cells in Rats

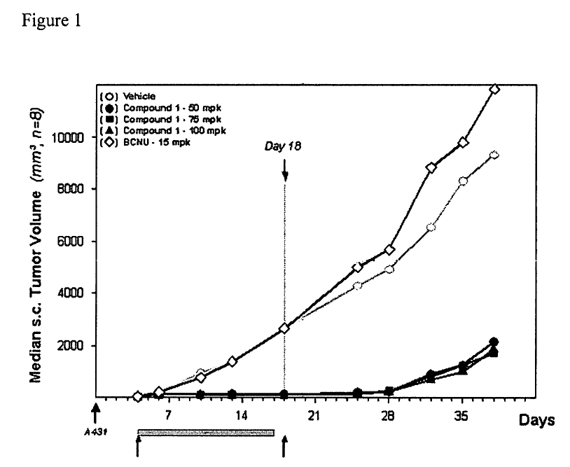

At the three doses tested, i.e. 50 mpk, 75 mpk and 100 mpk, no clinical signs of toxicity such as body weight loss or behavioral changes were associated to the treatment with MTKI 1. Looking at the differences in median subcutaneous tumor volumes observed in the different animals (FIG. 1), there was no effect of BCNU on A431 tumor growth, whereas MTKI 1 clearly inhibited subcutaneous A431 tumor growth at all doses tested. The high dose of BCNU (15 mpk) may have induced some mild toxicity (evident from sooner appearance of clinical signs of toxicity, i.e. body weight loss) and explains an even increased tumor growth rate when compared to the vehicle controls.

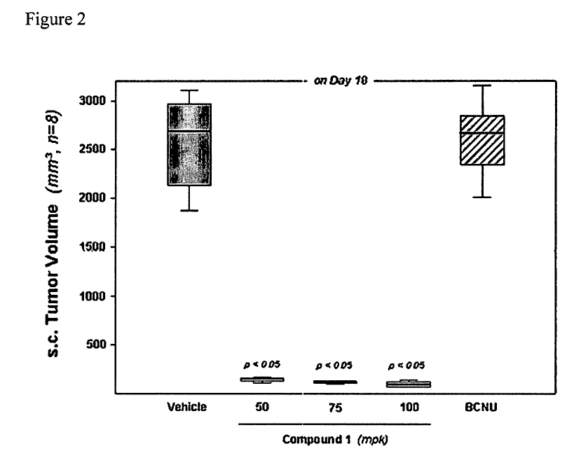

On day 18, i.e. 24 hr after the last MTKI 1 treatment cycle, statistical analysis of the s.c. A431 tumors (FIG. 2) showed a significant effect of MTKI 1 on s.c. tumor growth at all doses tested. Treatment versus Control (taking control as 100%) values were 5.37% for 50 mpk of MTKI 1, 4.29% for 75 mpk of MTKI 1 and 3.17% for 100 mpk of MTKI 1.

### Stereotaxic Injection of Cells in the Brain of Rats

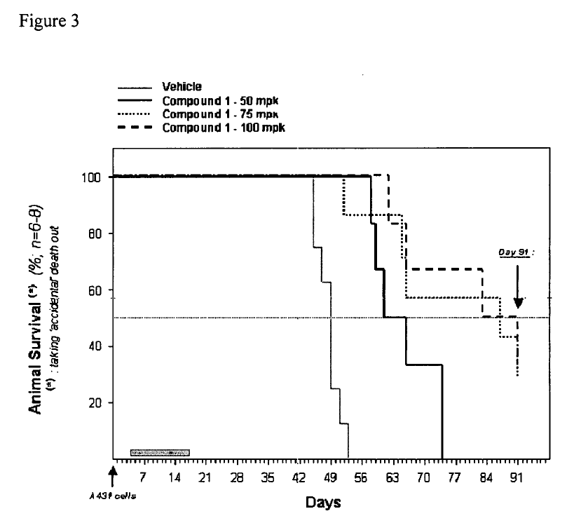

The Kapalan Myer survival curves (FIG. 3) clearly show the survival benefit for animals treated with MTKI 1 (Compound 1). A dose-dependent marked effect of MTKI 1 (Compound 1) on animal survival time (few ‘accidental’ animal death were observed following documented mis-administration of the compound in the lungs, for instance, and/or not related to the presence of brain tumors) was observed. Survival times (50%) changed from 49 for vehicle treated animals to 61, 83 and 88 days for animals treated with 50 mpk, 75 mpk and 100 mpk of Compound 1 respectively.

In these experiments, some animals surviving at day 91 had to be sacrificed due to the extensive s.c. tumor burden.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method for the treatment or prevention of brain cancer or brain cancer metastases in a mammalian subject, comprising administering a therapeutically effective amount of a compound chosen from the group consisting of 4,6-ethanediylidenepyrimido[4,5-b][6,1,12]benzoxadiazacyclo-pentadecine, 17-bromo-8,9,10,11,12,13,14,19-octahydro-20-methoxy-13-methyl or a pharmaceutically acceptable acid or base addition salt thereof; or 17-bromo-8,9,10,11,12,13,14,19-octahydro-20-methoxy-13-methyl-4,6-ethanediylidenepyrimido [4,5-b][6,1,12]benzoxadiazacyclo pentadecine dihydrobromide to a mammalian subject in need of such treatment.

2. The method of claim 1, in which the compound has the following structure:

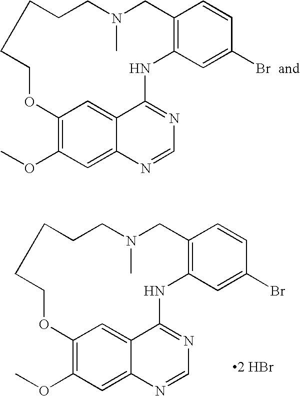

3. The method as claimed in claim 1 wherein a therapeutically effective amount of the compound is administered orally, parenterally, topically or intrathecally.

4. The method as claimed in claim 1 wherein 4,6-ethanediylidenepyrimido[4,5-b][6,1,12]benzoxadiazacyclo-pentadecine, 17-bromo-8,9,10,11,12,13,14,19-octahydro-20-methoxy-13-methyl or a pharmaceutically acceptable acid or base addition salt thereof, is administered in combination with a further anti-cancer agent.

5. The method as claimed in claim 4, wherein the further anti-cancer agent is selected from the group consisting of Temozolomide and BCNU.
